(19) United States
(12) Patent Application Publication
(10) Pub. No.: US 2008/0248099 A1
(43) Pub. Date: Oct. 9, 2008

(54) Method for Treating Disease or Disorder of Adult Central Nervous System Associated with Tissue Shrinkage or Atrophy by Administration of Insulin

(76) Inventor: Douglas N. Ishii, La Porte, CO (US)

Correspondence Address: HOWREY LLP-HN, C/O IP DOCKETING DEPARTMENT, 2941 FAIRVIEW PARK DRIVE, SUITE 200, FALLS CHURCH, VA, 22042-7195, US

(21) Appl. No.: 12/093,249

(22) PCT Filed: Nov. 10, 2006
(86) PCT No.: PCT/US2006/043667

§ 371 Date: May 9, 2008

Related U.S. Application Data
(60) Provisional application No. 60/735,606, filed on Nov. 11, 2005.

(51) Int. Cl. A61K 9/127; C07K 14/62; A61K 38/28; A61K 31/711; A61P 25/28; A61K 31/17; A61K 9/00; A61K 48/00; A61P 25/16; A61P 3/10

(52) U.S. Cl. 424/450; 530/303; 514/3; 514/44; 424/93.21; 424/484; 514/592

(57) ABSTRACT

The present invention provides a use of an insulin for the manufacture of a medicament for treating or preventing diseases or disorders of adult brain. Also provided is a method for treating or preventing diseases or disorders of adult brain by administering an effective amount of an insulin to the brain. The diseases or disorders of adult brain are associated with tissue shrinkage or atrophy. The amount of the insulin administered is preferably from 0.001 Units per kg body weight per day up to 10 Units per kg body weight per day.

This application claims the benefit under 35 U.S.C. 119(e) of U.S. Provisional Application Ser. No. 60/735,606, filed Nov. 11, 2005, the entire contents of which are incorporated herein by reference.

### GOVERNMENT FUNDING

This invention was made partly through the grant R49/CR811509 from Centers for Disease Control and Injury. Therefore, the U.S. government has certain rights in this invention.

### BACKGROUND OF THE INVENTION

1. Field of the Invention

The present invention generally relates to medical treatment. More specifically, the present invention relates to use of an insulin for manufacture of a medicament for treating a disease or disorder of a central nervous system of an adult individual and method of treating such disease or disorder using an insulin or composition that enhances the activity of endogenous insulin. Such disease or disorder is associated with tissue shrinkage or atrophy.

2. Description of the Related Art

Many people suffer from disorders and diseases of the brain in which there is significant tissue shrinkage, loss, atrophy or cell death. Such atrophy may be associated with loss of tissue wet weight, dry weight, protein, DNA and/or cells. Such diseases and disorders may include Alzheimer's disease, brain atrophy associated with diabetes and dementia (diabetic dementia), Parkinson's disease, Huntington's Disease, senile dementia, multiple sclerosis, dementia associated with Acquired Immunodeficiency Syndrome (AIDS), Pick's Disease, stroke, trauma, diffuse cerebral sclerosis of Schilder, acute necrotizing hemorrhagic encephalomyelitis, cortical-basal ganglionic syndromes, familial dementia, and progressive supranuclear palsy. MRI or PET scans have been used to show loss of brain mass or brain shrinkage, for example, in Alzheimer's disease, diabetic dementia, Parkinson's disease, multiple sclerosis, dementia associated with AIDS, and senile or familial dementia. In stroke, trauma, Alzheimer's disease and diabetic dementia the extent of loss of brain cells may be variable depending on severity and duration of the insult. These diseases and disorders of the brain are well documented in various textbooks of neurology.

It would be clinically useful if the factors that normally regulated adult brain weight were more completely understood. Such factors might be useful in treating diseases or disorders in which there is brain tissue atrophy, loss of tissue wet weight, dry weight, protein and cells. It is know, for example, that certain neurotrophic factors such as nerve growth factor (NGF) can support brain cell survival. However, NGF only acts on cells containing the Trk A receptor, primarily the cholinergic neurons in the brain, and NGF's action is restricted to the small fraction of brain cells that are cholinergic. It would obviously be desirable to identify neurotrophic factors that acted more broadly on the many neuron types in the brain.

It is known that insulin is present in the brain and that insulin receptors are widely distributed throughout the brain. However, the role of insulin in the adult mammalian brain beyond the regulation of satiety and body weight has been poorly understood. Effects of insulin were discussed previously (Recio-Pinto and Ishii, 1988), including the distribution of insulin in the brain, its effects on feeding behavior, electrical activities of neurons, and neuromodulation. The effects of insulin on synapses, neuron survival, neurite outgrowth, and protein, RNA and DNA contents of cultured embryonic cells were further discussed in Recio-Pinto and Ishii (1988). However, it is appreciated in the art that embryonic neurons and adult neurons often do not respond, or respond differently, to the same factors. Further, it is recognized that responses in cell culture are not predictive of effects in vivo. Prior to the present invention, it was not known whether insulin could prevent brain atrophy or tissue loss, particularly in the adult mammal or whether insulin can directly regulate brain weight, atrophy or tissue loss in diabetic brain disorders.

In fact, a conceptual impediment to understanding the direct effect of insulin on the brain has inhibited progress in the field. In diabetes, MRI shows brain shrinkage or atrophy in both Type 1 and Type 2 diabetic patients (Lunetta et al., 1994; Dejgaard et al., 1991; Araki et al., 1994). Such atrophy is independent of cerebrovascular disease (Araki et al., 1994). It is very widely believed by clinicians that neurological complications in diabetes are due to hyperglycemia (high blood glucose levels). Therefore, treatments with insulin, or treatments that increase its release from the pancreas or efficacy of insulin in the body, are believed to prevent neurological complications by reducing the hyperglycemia. However, this belief ignores the fact that such treatments alter two variables. Diabetic treatments with insulin, or with oral drugs that increase the release or efficacy of insulin, can increase signaling through the insulin receptor. Such increased signaling reduces hyperglycemia in diabetes. However, what is nearly universally overlooked is that such increased insulin receptor signaling at the same time can alter the expression of insulin responsive genes or processes that are unrelated to glucose regulation. Thus, two variables are changed as a result of insulin treatment. The possibility that insulin may directly prevent brain atrophy independently of hyperglycemia has not previously been investigated.

Impaired learning/memory when sufficiently severe can result in loss of capacity for self care, and patients with diabetic dementia (Ott et al., 1999), senile dementia, AIDS dementia or Alzheimer's disease become unable to dress, feed, bathe themselves or find their way back home. Dementia may also occur in Parkinson's disease. Nearly half of all patients in nursing homes have dementia. The diabetic rat is a model of a brain disease or disorder associated with brain atrophy and impaired learning/memory (Lupien et al., 2003). This is similar to the brain atrophy associated with cell loss observed in human diabetic and other dementias. It is particularly interesting that Alzheimer's disease is associated with brain insulin resistance involving reduced levels of brain insulin and reduced insulin signaling (Craft et al., 1998; Frolich et al., 1998). Thus, Alzheimer's disease shares with diabetes the brain atrophy, dementia and reduced insulin signaling, but not hyperglycemia.

Therefore, there exits a need for an effective method for treating diseases or disorders of a central nervous system of an adult individual, wherein the diseases or disorders are associated with tissue shrinkage or atrophy.

### SUMMARY OF THE INVENTION

The present invention is directed to a use of an insulin for manufacture of a medicament for treating or preventing a disorder or disease of a central nervous system of an adult individual, wherein the disorder or disease is associated with tissue shrinkage or atrophy.

The present invention is also directed to a method for treating or preventing a disorder or disease of a central nervous system of an adult individual, wherein the disorder or disease is associated with tissue shrinkage or atrophy. This method comprises administering to the adult individual with a pharmaceutically effective amount of an insulin.

The present invention is also directed to a method for treating or preventing a disorder or disease of a central nervous system of an adult individual, wherein the disorder or disease is associated with tissue shrinkage or atrophy, by administering intracranially or intrathecally to the adult individual with an insulin in an amount of from about 0.001 Units, more specifically, about 0.001 International Units (IU) per kg body weight per day to about 10 Units, more specifically, about 10 IU per kg body weight per day of an insulin.

The present invention is further directed to a method for treating or preventing a disorder or disease of a central nervous system of an adult individual, wherein the disorder or disease is associated with tissue shrinkage or atrophy. This method comprises administering to the adult individual with a pharmaceutically effective amount of composition that enhances the activity of endogenous insulin.

The foregoing and other advantages of the present invention will be apparent to those skilled in the art, in view of the following detailed description of the preferred embodiment of the present invention, taken in conjunction with the accompanying drawings.

### BRIEF DESCRIPTION OF THE DRAWINGS

Features of the present invention as well as a preferred mode of use, further objectives, and advantages thereof, will best be understood by reference to the following detailed description of an illustrative embodiment when read in conjunction with the accompanying drawings, wherein:

FIGS. 1A, 1B and 1C show that insulin prevents brain atrophy in accordance with the present invention.

FIGS. 2A and 2B show that insulin treatment demonstrated in FIGS. 1A-1C has no effect on hyperglycemia, yet partially prevents loss of body weights.

### DETAILED DESCRIPTION

The present invention is directed to a use of an insulin for manufacture of a medicament for treating or preventing a disorder or disease of a central nervous system of an adult individual as well as methods of treating such disease or disorder using an insulin or composition that enhances the activity of endogenous insulin. Such disease or disorder is associated with tissue shrinkage or atrophy.

In one embodiment of the present invention, there is provided a use of an insulin for manufacture of a medicament for treating or preventing a disorder or disease of a central nervous system of an adult individual, wherein the disorder or disease is associated with tissue shrinkage or atrophy. Representative examples of such disease or disorder include Alzheimer's disease, brain atrophy associated with diabetes, Parkinson's disease, Huntington's Disease, senile dementia, multiple sclerosis, dementia associated with Acquired Immunodeficiency Syndrome (AIDS), Pick's Disease, stroke, trauma, diffuse cerebral sclerosis of Schilder, acute necrotizing hemorrhagic encephalomyelitis, cortical-basal ganglionic syndromes, familial dementia, and progressive supranuclear palsy.

Particularly, the insulin can be human, beef, pork or fish insulin. Representative examples of the insulin that is useful in the present invention includes regular soluble insulin, Lispro insulin, neutral protamine Hagedorn (NPH) insulin, Lente insulin, Ultralente insulin, protamine zinc insulin or Glargine insulin. Still particularly, the above medicament further comprises a pharmaceutical excipient or adjuvant, for example, acetate, zinc, protamine, mannitol, glycine, or citrate, and is in a form for administration of the insulin in an amount of from about 0.001 Units, more specifically, about 0.001 International Units (IU) per kg body weight per day to about 10 Units, more specifically, about 10 IU per kg body weight per day. In the case of intracranial or intrathecal administration, the insulin amount is preferably from about 0.001 Units, more specifically, about 0.001 IU per kg body weight per day to about 5 Units, more specifically, about 5 IU per kg body weight per day.

In another embodiment of the present invention, there is provided a method for treating or preventing a disorder or disease of a central nervous system of an adult individual, wherein the disorder or disease is associated with tissue shrinkage or atrophy. This method comprises administering to the adult individual with a pharmaceutically effective amount of an insulin. Representative examples of such disease or disorder include Alzheimer's disease, brain atrophy associated with diabetes, Parkinson's disease, Huntington's Disease, senile dementia, multiple sclerosis, dementia associated with Acquired Immunodeficiency Syndrome (AIDS), Pick's Disease, stroke, trauma, diffuse cerebral sclerosis of Schilder, acute necrotizing hemorrhagic encephalomyelitis, cortical-basal ganglionic syndromes, familial dementia, and progressive supranuclear palsy.

Particularly, the insulin is administered intracranially or intrathecally in an amount from about 0.001 Units, more specifically, about 0.001 IU per kg body weight per day to about 10 Units, more specifically, about 10 IU per kg body weight per day, and more preferably, from about 0.001 Units, more specifically, about 10 IU per kg body weight per day to about 5 Units, more specifically, about 5 IU per kg body weight per day. The unit dose of from 0.001 IU to 10 IU is approximately the same as from 30 nanograms to 0.3 milligrams insulin per kg body weight per day, because in highly purified insulin there is 25-30 IU per mg.

International Units (IU) are based on functional assays and defined by reference to Bangliam et al., (1978). Basically, IU is the amount of insulin that reduces glucose below a certain level within a certain time in a certain weight of rabbit or mouse. Since it is a functional unit, 1 IU of pork insulin has the same activity as 1 IU of insulin from another species. Insulin manufacturers routinely label their insulin strength in IU per ml.

Insulin preparations are defined in IU because different insulin preparations can vary in the number of milligrams needed per unit. Also, the insulin requirement among patients can vary, for example, based on weight, age, sex, level of exercise, meal frequency, and stress due to illness, surgery, trauma, or emotional duress. In the situation in which the insulin preparation is being delivered intracranially, intrathecally or otherwise directly into the central nervous system, the preferred dose would be approximately 0.1% to 6% of the total daily insulin units utilized or produced in the body per day by a patient.

For intracranial administration, a pump is used that releases the insulin through a catheter into a lateral ventricle of the brain. For intrathecal administration, insulin is delivered to the subarachnoid space or cisterna magna of the spinal cord.

Alternatively, the insulin can be administered intranasally in an amount of from about 30 Units, more specifically, about 30 IU to about 600 Units, more specifically, about 600 IU per day. In such intranasal delivery, the insulin is administered by way of transporting into the central nervous system at the blood-central nervous system-barrier (B-CNS-B) or through the local nasal circulatory system rather than through the olfactory neural pathway.

Besides the above routes of administration, the insulin can also be administered by injecting a vector that contains an insulin gene into the central nervous system, by transfecting a cell with an insulin gene and then transferring the transfected cell into the central nervous system, by encapsulating the insulin into liposomes and then delivering the liposomes to the central nervous system, or by preparing the insulin in a matrix and then implanting the matrix into the central nervous system. The insulin preparations may be used as single preparations or as mixtures, and/or the administration of insulin may be continuous or intermittent.

Particularly, the insulin can be human, beef, pork or fish insulin such as regular soluble insulin, Lispro insulin, neutral protamine Hagedorn (NPH) insulin, Lente insulin, Ultralente insulin, protamine zinc insulin or Glargine insulin.

In still another embodiment of the present invention, there is provided a method for treating or preventing a disorder or disease of a central nervous system of an adult individual, wherein the disorder or disease is associated with tissue shrinkage or atrophy, by administering intracranially or intrathecally to the adult individual with an insulin in an amount of from about 0.001 Units, more specifically, about 0.001 IU per kg body weight per day to about 10 Units, more specifically, about 10 IU per kg body weight per day, and preferably, from about 0.001 Units, more specifically, about 0.001 IU per kg body weight per day to about 5 Units, more specifically, about 5 IU per kg body weight per day.

In yet another embodiment of the present invention, there is provided a method for treating or preventing a disorder or disease of a central nervous system of an adult individual, wherein the disorder or disease is associated with tissue shrinkage or atrophy. This method comprises administering to the adult individual with a pharmaceutically effective amount of composition that enhances the activity of endogenous insulin. Representative examples of such disorder or disease include Alzheimer's disease, brain atrophy associated with diabetes, Parkinson's disease, Huntington's Disease, senile dementia, multiple sclerosis, dementia associated with Acquired Immunodeficiency Syndrome (AIDS), Pick's Disease, stroke, trauma, diffuse cerebral sclerosis of Schilder, acute necrotizing hemorrhagic encephalomyelitis, cortical-basal ganglionic syndromes, familial dementia or progressive supranuclear palsy. Also particularly, molecules like tolbutamide, chlorpropamide, tolazamide, acetohexamide, glyburide, glipizide, gliclazide, glimepiride and metformin are suitable for the present invention.

The present study demonstrates for the first time that insulin is a broadly acting neurotrophic factor and can treat diseases or disorders of the brain or spinal cord, including those in which there is tissue shrinkage, atrophy, and loss of brain or spinal cord matter. Examples of such diseases or disorders include Alzheimer's disease, brain atrophy associated with diabetes and dementia (diabetic dementia), Parkinson's disease, Huntington's Disease, senile dementia, multiple sclerosis, dementia associated with Acquired Immunodeficiency Syndrome (AIDS), Pick's Disease, stroke, trauma, diffuse cerebral sclerosis of Schilder, acute necrotizing hemorrhagic encephalomyelitis, cortical-basal ganglionic syndromes, familial dementia, and progressive supranuclear palsy. The data suggest that insulin may be used to manufacture a medicament for treating diseases or disorders of the brain or spinal cord by administration of insulin in a manner that increases insulin concentrations within these tissues. In the case of nondiabetic patients, insulin administered into the peripheral circulation may cause unwanted and potentially dangerous hypoglycemia, including confusion, coma, convulsions and potentially death. Routes of insulin administration to the brain that avoid or minimize the risk of hypoglycemia are studied using an animal model of brain disease or disorder with brain atrophy and loss of brain mass.

The present study also demonstrates that insulin is effective in preventing or ameliorating brain diseases or disorders in a variety of conditions in which there is brain tissue atrophy or loss, including Alzheimer's disease, brain atrophy associated with diabetes and dementia (diabetic dementia), Parkinson's disease, Huntington's Disease, senile dementia, multiple sclerosis, dementia associated with Acquired Immunodeficiency Syndrome (AIDS), Pick's Disease, stroke, trauma, diffuse cerebral sclerosis of Schilder, acute necrotizing hemorrhagic encephalomyelitis, cortical-basal ganglionic syndromes, familial dementia, and progressive supranuclear palsy.

For example, the stroke may be due to cerebrovascular accidents or hypoxic-ischemic episodes. Diseases associated with brain atrophy or degeneration may include lobar atrophy, microcephaly, hydrocephaly, Wemicke-Korsakoff syndrome, Niemann-Pick disease, Gaucher's disease, leukodystrophy, or Fabry's disease.

The present study further demonstrates that insulin is effective in treating an animal model of dementia, which suggests that insulin may be used to treat brain diseases or disorders where there may be tissue or cell loss associated with dementia including diabetic dementia, Alzheimer's disease, Parkinson's disease, AIDS dementia, and learning and memory disorders associated with stroke and trauma.

Insulins can be produced by recombinant DNA technology. The complete amino acid sequence of insulin from various species is known, including human, beef, porcine and fish. Animal insulins may be purified from tissues or made from recombinant cDNA. Lispro is an analog of human insulin in which the amino acid residues at B28 and B29 are reversed. Aspart insulin is also a human insulin analog in which aspartic aid is replaced for proline at B28. Neutral protamine Hagedorn is NPH insulin, also known as isophane insulin suspension. Lente insulin is insulin zinc suspension. Ultralente is crystallized, whereas semilente is amorphous insulin in an acetate buffer. Protamine zine insulin is a complex containing protamine and zinc that extends the half-life of insulin. Glargine insulin is human insulin in which two arginine residues are added to the C terminus of the B chain, and glycine replaces asparagine at position A21 on the A chain. The various preparations of shorter and longer acting insulins may be mixed to modify the duration of insulin action. These human and animal insulins are commercially available and have been used to treat human patients, and their methods of purification and preparation are known in the art. The insulin formulations may contain various pharmaceutical excipients or adjuvants to stabilize, buffer, increase half-life or otherwise enhance insulin-containing medicaments. The excipients or adjuvants may include but are not limited to acetate, zinc, protamine, mannitol, glycine, or citrate. The normal insulin production in a healthy thin adult human is approximately 0.5 IU per kg body weight per day, whereas obese Type 2 diabetic patients require about 2 IU per kg body weight per day due to insulin resistance.

The preferred routes of insulin administration in accordance with the present invention are intended to deliver insulin at an effective dose within the central nervous system while at the same time avoiding undesirable hypoglycemia. Not only must the insulin (e.g. a formulation of an insulin or a vector comprising an insulin gene) be able to cross the blood-central nervous system-barrier (B-CNS-B), it must also exist, or in the case of a vector comprising an insulin gene, such insulin gene must be expressed in a sufficient amount at the diseased site to treat the disease, and in the mean time, the amount of the insulin delivered or expressed at the diseased site must not be excessive in order to avoid toxicity. The toxicity may include potentially fatal hypoglycemia if the insulin exists in excessive amounts.

Various routes of administration may be used to treat disorders and diseases of the central nervous system with an insulin. For example, intracranial administration may include insulin infusion into the lateral brain ventricles from a catheter connected to a pump driven by mechanical or osmotic forces. Insulin may also be infused intrathecally into the subarachnoid space to treat the spinal cord. Other possible routes of administration include cloning the insulin gene into a suitable vector under the control of a suitable promoter and then directly injecting the vector into the brain or spinal cord. Alternatively, the vector comprising the insulin gene may first be transfected into cells (including the patient's cells), and such cells then be transferred into the brain or spinal cord for the long-term production of insulin within the central nervous system. Another route of administration is preparing insulin in a matrix and then implanting the matrix into the central nervous system to slowly release insulin. Insulin may also be incorporated or encapsulated into liposomes, and the liposomes injected to deliver the insulin across the blood-central nervous system-barrier (B-CNS-B) which includes blood-brain-barrier (BBB) or blood-spinal cord-barrier (B-SC-B). Still another possible route is that insulin may be administered intranasally, wherein the high local concentration of insulin in the highly vascularized nasal compartment can deliver insulin efficiently across the blood-brain-barrier into the cerebrovascular fluid. The intranasal insulin would become diluted in the systemic circulation, thereby avoiding hypoglycemia. The intranasal insulin may be formulated as a liquid, suspension or powder. When administered intranasally, the amount of insulin needs to be higher compared to intracranial or intrathecal delivery due to incomplete uptake. The preferred effective dose is between about 30 to about 600 IU per day delivered in three or four divided doses.

On the other hand, subcutaneous or intramuscular routes of insulin administration are not preferred. These routes of administration may reduce the systemic concentration of glucose and potentially cause hypoglycemia, particularly in nondiabetic elderly patients.

Through the preferred routes of administration discussed above, small composition that enhances the activity of endogenous insulins can also be administered to the central nervous system to prevent atrophy, shrinkage or tissue loss in diseases or disorders of the central nervous system. Such small molecules include tolbutamine, chlorpropamide, tolazamide, acetohexamide, glyburide, glipizide, gliclazide, glimepiride or metformin. These molecules have been prepared commercially and their methods of manufacture are known in the art. Such small molecules should not be used in diabetic dementia associated with Type 1 diabetes, because amounts of insulin are produced in this disorder are too insufficient for these small molecules to be effective. Such small molecules may, however, be used in elderly nondiabetic subjects or those with Type 2 diabetes.

The following examples are given for the purpose of illustrating various embodiments of the invention and are not meant to limit the present invention in any fashion.

### EXAMPLE

### Materials and Methods

Adult rats were randomly assigned to groups and one group was treated with streptozotocin to induce diabetes (nondiabetic, 12 rats; diabetic, 9 rats). Streptozotocin binds to and destroys the beta cells of the pancreas resulting in the inability to produce insulin. After 12 weeks, rats were euthanized, and brains excised by single knife cut at rostral edge of cerebellum. Wet weights were determined, brains homogenized in a buffer, rapidly frozen in liquid nitrogen, and stored at −70° C. Aliquots were dried in a lyophilizer to determine dry weights. Aliquots were used to determine protein content per brain using a dye-binding assay, and DNA content per brain was determined using Hoechst dye 33258 and fluorometry (Salmon sperm DNA was used for standard concentration curves). Values are group means. The CSS Statistica software package was used to calculate Newman-Keuhls posthoc test of means.

Table 1 shows that there is a significant loss of brain wet weight, dry weight, DNA and protein in a rat model of brain atrophy associated with dementia. The tissue dry weight is comprised of all of the non-water components of tissues, including DNA, RNA, protein, lipids, and small molecules. The brain atrophy is shown further to be associated with a significant decrease in brain protein content as well as brain DNA content. There are approximately 100 billion cells in the rat brain. The loss of 9% of brain DNA or approximately 9 billion cells shows that there is a significant cell loss. In rats that were treated identically, learning and memory was found to be significantly impaired in the diabetic vs. nondiabetic rats in a Morris Water Maze (Lupien et al., 2003). Thus, brain atrophy is associated with impaired cognitive function in this model of brain disease and disorder.

TABLE 1

| Brain Parameter | Nondiabetic | Diabetic | P value |
|---|---|---|---|
| Wet weight (g) | 2.16 | 1.82 | <0.001 |
| Water (g) | 1.66 | 1.42 | <0.001 |
| Dry (mg) | 405 | 322 | <0.001 |
| DNA (mg) | 1.41 | 1.28 | <0.03 |
| Protein (mg) | 222 | 179 | <0.001 |

### Effects of Insulin in Treating Brain Atrophy

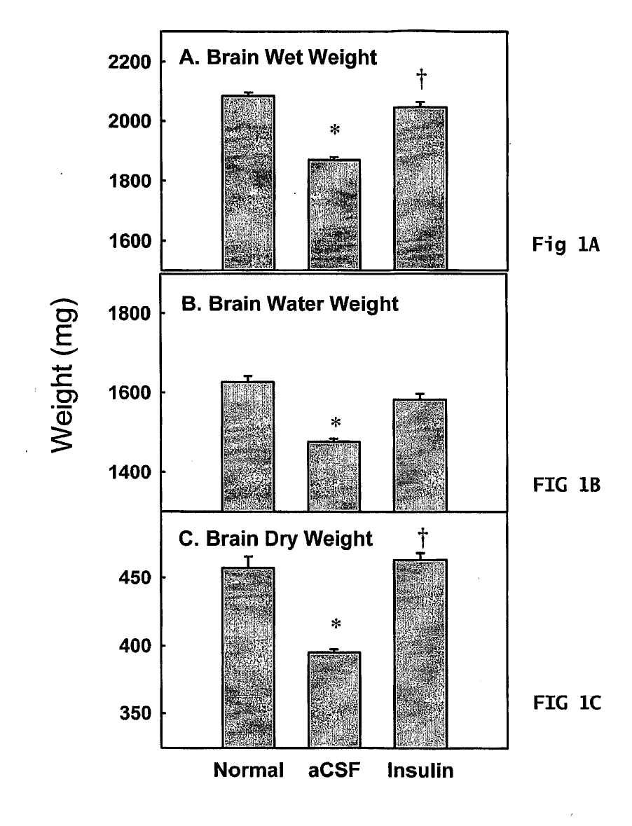

Adult rats were randomly assigned to treatment groups, some of which were treated with streptozotocin to induce diabetes. Alzet osmotic minipumps (pump rate, 0.5 μl per hour) were connected to catheters that delivered either artificial cerebrospinal fluid (D+aCSF) or 0.3 U insulin per kg body weight per day (D+Insulin) into the brain lateral ventricles of diabetic rats for 10 weeks. Pumps were replaced every two weeks. Brains were removed and the wet weight determined. The brains were homogenized in a buffer, and aliquots were taken to determine dry weight. The water weight was calculated as the difference between brain wet and dry weights. The results are shown in FIGS. 1A-1C, wherein the values are means ±SEM (N=7 rats per group). Newman-Keuls posthoc test of means was calculated using CSS Statistica software package.

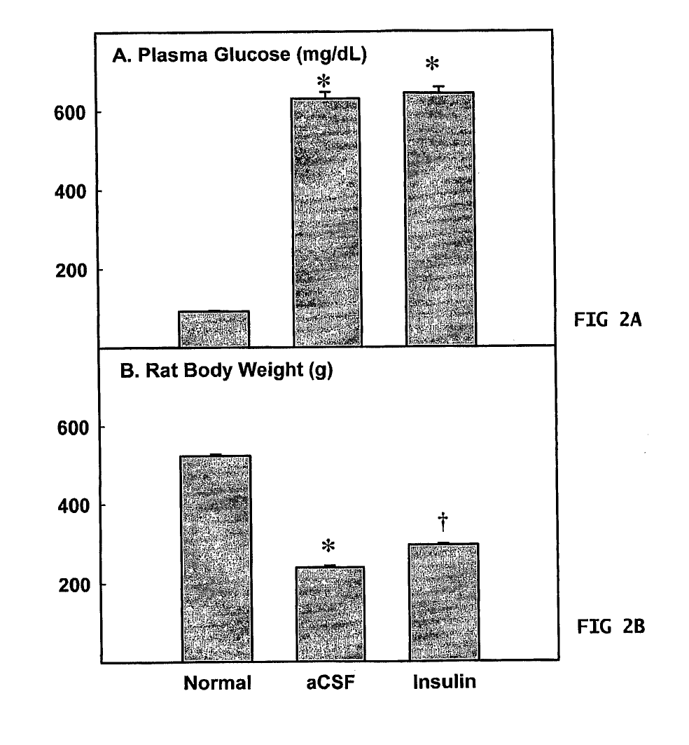

*P<0.01 for Nondiabetic vs. D+aCSF groups suggests significant brain atrophy in the diabetic rats. P<0.02 for D+Insulin vs. D+aCSF groups suggests that insulin prevented brain atrophy, including the loss of brain wet and dry weights (see, FIGS. 1A-1C). Further, the dose of insulin administered was too small to reduce the hyperglycemia, as shown in FIG. 2A.

In the same experiment described in FIGS. 1A-1C, rats were weighed and tail blood was withdrawn for glucose assay prior to euthanasia at 10 weeks. Values are means ±SEM. Newman-Keuls posthoc test of means was calculated using CSS Statistica software package. FIGS. 2A and 2B demonstrate that the above-discussed insulin treatment had no effect on hyperglycemia, yet partially prevented loss of body weights. FIG. 2A shows serum glucose levels. *P<0.01 for Nondiabetic vs. D+aCSF or D+Insulin rats suggests significant hyperglycemia in both groups of diabetic rats. Nonsignificant p value for D+Insulin vs. D+aCSF rats indicates that the insulin treatment did not reduce hyperglycemia. FIG. 2B shows rat body weights. *P<0.01 for Nondiabetic vs. D+aCSF groups and P<0.02 for D+Insulin vs. D+aCSF groups suggest that insulin can partially prevent loss of body weight independently of hyperglycemia.

Further, the higher concentration of insulin within the brain completely normalized brain weight in diabetic rats. The insulin in cerebrospinal fluid (CSF) that exits the brain at the superior saggital sinus is released into the circulation and diluted; hence a very low insulin concentration exists outside the brain. Such low concentration of insulin only partially prevented loss of body weight (see, FIG. 2B) but was insufficient to reduce hyperglycemia (see, FIG. 2A).

The capacity of a low dose of insulin to partially prevent loss of body weight despite hyperglycemia is probably due to the capacity of insulin to directly regulate genes. In a glucose clamp experiment in which glucose levels were kept constant in patients, the infusion of insulin was found to regulate the expression of over 700 different genes in a muscle biopsy in a gene chip array experiment. Consequently, insulin can regulate gene expression in the body independently of glucose. The present study shows that the brain is also an insulin-responsive organ, independently of hyperglycemia. Insulin may well regulate the expression of a large number of genes in the brain to maintain brain weight.

### Discussion

The present study shows for the first time that insulin directly regulates brain weight of mammals, which suggests that the brain atrophy in diabetes is due to loss of a direct activity of insulin on the brain. The brain atrophy in Alzheimer's disease now may be understood as the consequence of reduced brain insulin signaling. In aging, a slow development of resistance to insulin may contribute to the slow shrinking of the brain and senile dementia. The present invention demonstrates that insulin treatment can prevent brain shrinkage or atrophy, which may further prevent the progression of brain deterioration.

Having revealed the capacity of insulin to prevent brain atrophy and support brain cells, an experiment to study the effect of insulin in the spinal cord can be similarly conducted by infusing insulin intrathecally into the spinal cord of adult diabetic rats under conditions that do not reduce hyperglycemia. Administration of insulin in an amount from 0.001 IU per kg body weight per day up to 10 IU per kg body weight per day is expected to be effective in treating spinal cord diseases or disorders. Studies have established that there is spinal cord atrophy with loss of dry weight, DNA and protein in diabetic rats. Insulin is likely to be found to prevent such spinal cord atrophy, tissue shrinkage, tissue loss, and cell loss independently of hyperglycemia. Without departing from the scope of the present invention, insulin can be used to treat spinal cord diseases or disorders, including traumatic injuries and amyotrophic lateral sclerosis.

While the invention has been shown in only a few of its forms, it should be apparent to those skilled in the art that it is not so limited but susceptible to various changes without departing from the scope of the invention.

1. A use of an insulin for manufacture of a medicament for treating or preventing a disorder or disease of a central nervous system of an adult individual, wherein said disorder or disease is associated with tissue shrinkage or atrophy and is selected from the group consisting of Alzheimer's disease, brain atrophy associated with diabetes, Parkinson's disease, Huntington's Disease, senile dementia, multiple sclerosis, dementia associated with Acquired Immunodeficiency Syndrome (AIDS), Pick's Disease, trauma, diffuse cerebral sclerosis of Schilder, acute necrotizing hemorrhagic encephalomyelitis, cortical-basal ganglionic syndromes, familial dementia, and progressive supranuclear palsy.

2. (canceled)

3. The use of claim 1, wherein said medicament further comprises a pharmaceutical excipient or adjuvant.

4. The use of claim 3, wherein said pharmaceutical excipient or adjuvant is acetate, zinc, protamine, mannitol, glycine, or citrate.

5. The use of any of claims 1, 3 and 4, wherein said insulin is human, beef, pork or fish insulin.

6. The use of any of claims 1, 3 and 4, wherein said insulin is regular soluble insulin, Lispro insulin, neutral protamine Hagedorn (NPH) insulin, Lente insulin, Ultralente insulin, protamine zinc insulin or Glargine insulin.

7. The use of any of claims 1, 3 and 4, wherein said medicament is in a form for administration of said insulin in an amount of from about 0.001 International Units (IU) per kg body weight per day to about 10 IU per kg body weight per day.

8. The use of claim 7, wherein said medicament is in a form for administration of said insulin in an amount of from about 0.001 IU per kg body weight per day to about 5 IU per kg body weight per day.

9. A method for treating or preventing a disorder or disease of a central nervous system of an adult individual, wherein said disorder or disease is associated with tissue shrinkage or atrophy and is selected from the group consisting of Alzheimer's disease, brain atrophy associated with diabetes, Parkinson's disease, Huntington's Disease, senile dementia, multiple sclerosis, dementia associated with Acquired Immunodeficiency Syndrome (AIDS), Pick's Disease, trauma, diffuse cerebral sclerosis of Schilder, acute necrotizing hemorrhagic encephalomyelitis, cortical-basal ganglionic syndromes, familial dementia, and progressive supranuclear palsy, comprising administering to said adult individual with a pharmaceutically effective amount of an insulin through a non-nasal route.

10. (canceled)

11. The method of claim 9, wherein said insulin is administered intracranially or intrathecally in an amount of from about 0.001 IU per kg body weight per day to about 10 IU per kg body weight per day.

12. The method of claim 11, wherein said insulin is administered intracranially or intrathecally in an amount of from about 0.001 IU per kg body weight per day to about 5 IU per kg body weight per day.

13. The method of claim 11 or 12, wherein said insulin is administered intracranially by way of a pump that releases said insulin through a catheter into a lateral ventricle of the brain of said adult individual.

14. The method of claim 11 or 12, wherein said insulin is administered intrathecally into the subarachnoid space or cisterna magna of the spinal cord of said adult individual.

15-16. (canceled)

17. The method of claim 9, wherein said insulin is administered by injecting a vector that contains an insulin gene into the central nervous system of said adult individual.

18. The method of claim 9, wherein said insulin is administered by transfecting a cell with an insulin gene and then transferring the transfected cell into the central nervous system of said adult individual.

19. The method of claim 9, wherein said insulin is administered by encapsulating said insulin into liposomes and then delivering said liposomes to the central nervous system of said adult individual.

20. The method of claim 9, wherein said insulin is administered by preparing said insulin in a matrix and then implanting said matrix into the central nervous system of said adult individual.

21. The method of claim 9, wherein said insulin is human, beef, pork or fish insulin.

22. The method of claim 9, wherein said insulin is regular soluble insulin, Lispro insulin, neutral protamine Hagedorn (NPH) insulin, Lente insulin, Ultralente insulin, protamine zinc insulin or Glargine insulin.

23. A method for treating or preventing a disorder or disease of a central nervous system of an adult individual, wherein said disorder or disease is associated with tissue shrinkage or atrophy and is selected from the group consisting of Alzheimer's disease, brain atrophy associated with diabetes, Parkinson's disease, Huntington's Disease, senile dementia, multiple sclerosis, dementia associated with Acquired Immunodeficiency Syndrome (AIDS), Pick's Disease, trauma, diffuse cerebral sclerosis of Schilder, acute necrotizing hemorrhagic encephalomyelitis, cortical-basal ganglionic syndromes, familial dementia, and progressive supranuclear palsy, comprising administering intracranially or intrathecally to said adult individual with an insulin in an amount of from about 0.001 IU per kg body weight per day to about 10 IU per kg body weight per day of an insulin.

24. The method of claim 23, wherein said insulin is administered in an amount of from about 0.001 IU per kg body weight per day to about 5 IU per kg body weight per day.

25. A method for treating or preventing a disorder or disease of a central nervous system of an adult individual, wherein said disorder or disease is associated with tissue shrinkage or atrophy and is selected from the group consisting of Alzheimer's disease, brain atrophy associated with diabetes, Parkinson's disease, Huntington's Disease, senile dementia, multiple sclerosis, dementia associated with Acquired Immunodeficiency Syndrome (AIDS), Pick's Disease, trauma, diffuse cerebral sclerosis of Schilder, acute necrotizing hemorrhagic encephalomyelitis, cortical-basal ganglionic syndromes, familial dementia, and progressive supranuclear palsy, comprising administering to said adult individual with a pharmaceutically effective amount of composition that enhances the activity of endogenous insulin.

26. (canceled)

27. The method of claim 25, wherein said composition is tolbutamide, chlorpropamide, tolazamide, acetohexamide, glyburide, glipizide, gliclazide, glimepiride or metformin.
